(19) United States
(12) Patent Application Publication
(10) Pub. No.: US 2025/0098998 A1
(43) Pub. Date: Mar. 27, 2025

(54) PSYCHOLOGICAL STATE ESTIMATION SYSTEM, PSYCHOLOGICAL STATE ESTIMATION METHOD, NON-TRANSITORY COMPUTER READABLE STORAGE MEDIUM

(71) Applicant: Oki Electric Industry Co., Ltd., Tokyo (JP)

(72) Inventor: Koji SAKURADA, Tokyo (JP)

(73) Assignee: Oki Electric Industry Co., Ltd., Tokyo (JP)

(21) Appl. No.: 18/758,885

(22) Filed: Jun. 28, 2024

(30) Foreign Application Priority Data
Sep. 25, 2023 (JP) 2023-160137

(51) Int. Cl. A61B 5/16; G16H 20/70

(52) U.S. Cl. CPC A61B 5/165; G16H 20/70

(57) ABSTRACT

To acquiring the subject's psychological progress related to the behavior change more easily and accurately. A psychological state estimation system includes an estimation unit configured to estimate, as psychological state data of a subject, at least one of motivation data, behavior change stage data, and self-efficacy data that are related to object behavior of the subject, on the basis of behavior data of the subject along time series.

### CROSS REFERENCE TO RELATED APPLICATION(S)

This application is based upon and claims benefit of priority from Japanese Patent Application No. 2023-160137, filed on Sep. 25, 2023, the entire contents of which are incorporated herein by reference.

### BACKGROUND

The present invention relates to a psychological state estimation system, a psychological state estimation method, and a non-transitory computer readable storage medium.

In recent years, the psychological phases related to behavior changes of a subject, until the health behavior or environmentally friendly behavior of the subject becomes a habit, may be represented by behavior change stages.

In the behavior change theory (TTM: transtheoretical model), which is widely used in the field of health, the states of awareness and behavior for object behavior are represented by five stages (a precontemplation period, a contemplation period, a preparation period, an action period, and a maintenance period). In TTM, each of these five stages is called a behavior change stage.

Specifically, the precontemplation period represents a stage in which the subject does not intend to change the behavior, and the contemplation period represents a stage in which the subject is interested in the behavior and intends to change the behavior within the next six months. Moreover, the preparation period represents a stage in which the subject intends to change the behavior within the next one month. The action period represents a stage in which the subject has changed the behavior within the last six months, and the maintenance period represents a stage in which the subject changed the behavior more than six months ago.

In TTM, it is considered that the utilization of a process of change (intervention method) suitable for the behavior change stages of a subject can contribute to the shift of the behavior change stage. The behavior change stage is represented by five stages. However, if the five behavior change stages are further divided into detailed phases in accordance with the psychological phases of the subject, the behavior change state of the subject may be divided into more phases, thereby improving the accuracy of intervention in accordance with the phases of the behavior change state.

For example, Japanese Patent Application Laid-open No. 2021-86280 discloses that the subject's phase (a planning phase, an action will phase, and an action phase) is determined in addition to the subject's behavior change stage, on the basis of the collection result of psychological states based on questionnaires conducted for a subject about once every several days, and the determination result of presence or absence of an action performed by the subject based on an questionnaire result or a sensing result of about once a day. These phases are applied only to the preparation period and the action period. Thus, the behavior change state is grasped in nine stages, and it is thus expected to exert the effect of improving the accuracy of intervention in accordance with the stages of the behavior change state.

### SUMMARY

However, in the technique disclosed in Japanese Patent Application Laid-open No. 2021-86280, it is necessary to collect a subject's psychological state by a questionnaire conducted once every several days, which increases a workload of the subject. Therefore, with the technique disclosed in Japanese Patent Application Laid-open No. 2021-86280, it is not possible to grasp the change in psychological state of the subject easily and accurately.

Moreover, in the technique disclosed in Japanese Patent Application Laid-open No. 2021-86280, the behavior change state is represented by stages and phases, but the phases are defined only in the preparation period and the action period. Thus, in the technique disclosed in Japanese Patent Application Laid-open No. 2021-86280, it is not possible to specifically grasp the change in psychological state of the subject whose behavior change state is a precontemplation period, a contemplation period (the precontemplation period and the contemplation period are also referred to as the “first half stage of behavior change”), or a maintenance period.

Therefore, the present invention mainly aims at solving the above-described problems and acquiring the subject's psychological progress related to the behavior change more easily and accurately.

In order to solve the above problems, according to one aspect of the present invention, a psychological state estimation system is provided. The psychological state estimation system includes an estimation unit configured to estimate, as psychological state data of a subject, at least one of motivation data, behavior change stage data, and self-efficacy data that are related to object behavior of the subject, on the basis of behavior data of the subject along time series.

The psychological state estimation system may include an intervention unit configured to intervene in the object behavior of the subject. The intervention unit may intervene by determining message data on the basis of the psychological state data and outputting the message data to a terminal of the subject.

The intervention unit may estimate a behavior level indicating a strength of the object behavior suitable for the subject, on the basis of the self-efficacy data, and determine message data corresponding to the behavior level.

The intervention unit may estimate the behavior level on the basis of a past behavior level and a target behavior level that are input by the subject, past self-efficacy data, and the self-efficacy data estimated by the estimation unit.

The estimation unit makes correction for decreasing the self-efficacy data in a case where subjects' behavior implementation frequency data of a first period is smaller than a predetermined first value, the behavior implementation frequency data indicating the number of days of implementation of the object behavior, and/or in a case where subject's message acceptability data regarding message data output to the terminal of the subject in the first period is smaller than a predetermined second value. The intervention unit may estimate the behavior level on the basis of self-efficacy data after the correction.

The behavior data may include service operation data that is operation data of the subject related to a service function for the subject. The estimation unit may estimate the motivation data on the basis of the service operation data.

The estimation unit may include a behavior analysis part configured to calculate service operation frequency data indicating a frequency of access to the service function by the subject during a first period, on the basis of the service operation data and a psychological state estimation part configured to estimate motivation data of the first period on the basis of the service operation frequency data.

The psychological state estimation part may estimate the motivation data of the first period to be larger as the service operation frequency data is larger.

The behavior data may include behavior implementation data including data indicating when the subject implemented the object behavior. The estimation unit may estimate the behavior change stage data on the basis of the behavior implementation data.

The estimation unit may include a behavior analysis part configured to calculate behavior implementation frequency data indicating the number of days of implementation of the object behavior by the subject during a first period, on the basis of the behavior implementation data and a psychological state estimation part configured to estimate behavior change stage data of the first period on the basis of the behavior implementation frequency data.

The estimation unit may estimate the motivation data. The psychological state estimation part may determine whether the number of days of implementation during the first period is smaller than a first number of days of implementation on the basis of the behavior implementation frequency data, determine, in a case where it is determined that the number of days of implementation during the first period is smaller than the first number of days of implementation, whether the motivation data is smaller than a first threshold, regard a value indicating a precontemplation period as the behavior change stage data in a case where it is determined that the motivation data is smaller than the first threshold, and regard a value indicating a contemplation period as the behavior change stage data in a case where it is determined that the motivation data is equal to or larger than the first threshold.

The psychological state estimation part may determine whether the number of days of implementation during the first period is equal to or larger than the first number of days of implementation and smaller than a second number of days of implementation on the basis of the behavior implementation frequency data, and regard a value indicating a preparation period as the behavior change stage data in a case where it is determined that the number of days of implementation during the first period is equal to or larger than the first number of days of implementation and smaller than the second number of days of implementation.

The psychological state estimation part may determine whether the number of days of implementation during the first period is equal to or larger than the second number of days of implementation on the basis of the behavior implementation frequency data, determine, in a case where it is determined that the number of days of implementation during the first period is equal to or larger than the second number of days of implementation, whether a continued period after an action period of the object behavior by the subject is smaller than a second threshold, regard a value indicating an action period as the behavior change stage data in a case where it is determined that the continued period after the action period is smaller than the second threshold, and regard a value indicating a maintenance period as the behavior change stage data in a case where it is determined that the continued period after the action period is equal to or larger than the second threshold.

The behavior data may include behavior implementation data including data indicating when the subject implemented the object behavior. The estimation unit may estimate the self-efficacy data on the basis of the behavior implementation data.

The estimation unit may include a behavior analysis part configured to calculate behavior implementation frequency data indicating the number of days of implementation of the object behavior by the subject during a first period, on the basis of the behavior implementation data and a psychological state estimation part configured to estimate the self-efficacy data of the first period on the basis of the behavior implementation frequency data.

The estimation unit may estimate the motivation data. The psychological state estimation part may determine whether the number of days of implementation during the first period is smaller than a first number of days of implementation on the basis of the behavior implementation frequency data, and estimate, in a case where it is determined that the number of days of implementation during the first period is smaller than the first number of days of implementation, self-efficacy data of the first period on the basis of a tendency of increase or decrease of motivation data from a second period that is a period before the first period to the first period and the self-efficacy data of the second period.

The estimation unit may estimate the motivation data. The psychological state estimation part may determine whether the number of days of implementation during the first period is equal to or larger than a first number of days of implementation and smaller than a second number of days of implementation on the basis of the behavior implementation frequency data, and estimate, in a case where it is determined that the number of days of implementation during the first period is equal to or larger than the first number of days of implementation and smaller than the second number of days of implementation, self-efficacy data of the first period on the basis of a tendency of increase or decrease of at least one of motivation data and behavior implementation frequency data from a second period that is a period before the first period to the first period and the self-efficacy data of the second period.

The psychological state estimation part may determine whether the number of days of implementation during the first period is equal to or larger than the second number of days of implementation on the basis of the behavior implementation frequency data, and estimate, in a case where it is determined that the number of days of implementation during the first period is equal to or larger than the second number of days of implementation, self-efficacy data of the first period on the basis of a tendency of increase or decrease of the behavior implementation frequency data from a second period that is a period before the first period to the first period and the self-efficacy data of the second period.

In order to solve the above problems, according to another aspect of the present invention, a psychological state estimation method is provided. The psychological state estimation method includes Estimating by a processor, as psychological state data of a subject, at least one of motivation data, behavior change stage data, and self-efficacy data that are related to object behavior of the subject, on the basis of behavior data of the subject along time series.

In order to solve the above problems, according to another aspect of the present invention, a non-transitory computer readable storage medium recording a program is provided. The program causes a computer to function as an estimation unit configured to estimate, as psychological state data of a subject, at least one of motivation data, behavior change stage data, and self-efficacy data that are related to object behavior of the subject, on the basis of behavior data of the subject along time series.

As described above, the present invention provides a technique capable of acquiring the subject's psychological progress related to the behavior change more easily and accurately.

### BRIEF DESCRIPTION OF THE DRAWINGS

FIG. 1 is a diagram illustrating a configuration example of a psychological state estimation system according to an embodiment of the present invention;

FIG. 2 is a diagram for explaining the operation of a psychological state estimation system 1 according to an embodiment of the present invention;

FIG. 3 is a diagram illustrating examples of terminal response data;

FIG. 4 is a diagram illustrating examples of behavior analysis data;

FIG. 5 is a diagram illustrating examples of psychological state data;

FIG. 6 is a diagram illustrating examples of behavior levels and behavior content images;

FIG. 7 is an example of message data held by an intervention unit 130; and

FIG. 8 is a diagram illustrating a hardware configuration of an information processing apparatus as an example of the hardware configuration of a server 10 according to an embodiment of the present invention.

### DETAILED DESCRIPTION OF THE EMBODIMENT(S)

Hereinafter, referring to the appended drawings, preferred embodiments of the present invention will be described in detail. It should be noted that, in this specification and the appended drawings, structural elements that have substantially the same function and structure are denoted with the same reference numerals, and repeated explanation thereof is omitted. 0. Overview First, the overview of an embodiment of the present invention will be described.

The embodiment of the present invention mainly proposes the technique of acquiring the subject's psychological progress related to the behavior change more easily and accurately. Note that the subject's psychological progress related to the behavior change is acquired as psychological state data indicating the psychological state of the subject. Examples of the psychological state data include data indicating behavior change stages, data indicating self-efficacy, and data indicating motivation. The behavior change stages, the self-efficacy, the motivation, and the like will be described later in detail.

In the following description, the data indicating behavior change stages is also referred to as “behavior change stage data”. Moreover, the data indicating self-efficacy is also referred to as “self-efficacy data”. Furthermore, the data indicating motivation is also referred to as “motivation data”.

Specifically, the psychological state estimation system according to the embodiment of the present invention estimates, as psychological state data indicating a subject's psychological state, at least one of the behavior change stage data, self-efficacy data, and motivation data on the basis of the behavior data of the subject along the time series. With such estimation of psychological state data of the subject, it is possible to acquire the subject's psychological progress related to the behavior change more easily and accurately.

The overview of the embodiment of the present invention has been described.

### 1. Details of Embodiment

The following will describe the details of the embodiment of the present invention.

### 1-1. Description of Configuration

A configuration example of the psychological state estimation system according to an embodiment of the present invention will be described. A user of the psychological state estimation system according to the embodiment of the present invention (hereinafter, also simply referred to as a “user”) is a subject in need of making the object behavior a habit. The user implements the object behavior depending on a situation. The embodiment of the present invention mainly assumes that the object behavior is a use of stairs (hereinafter also referred to as “stair walking”). However, the object behavior is not limited to stair walking, as described later.

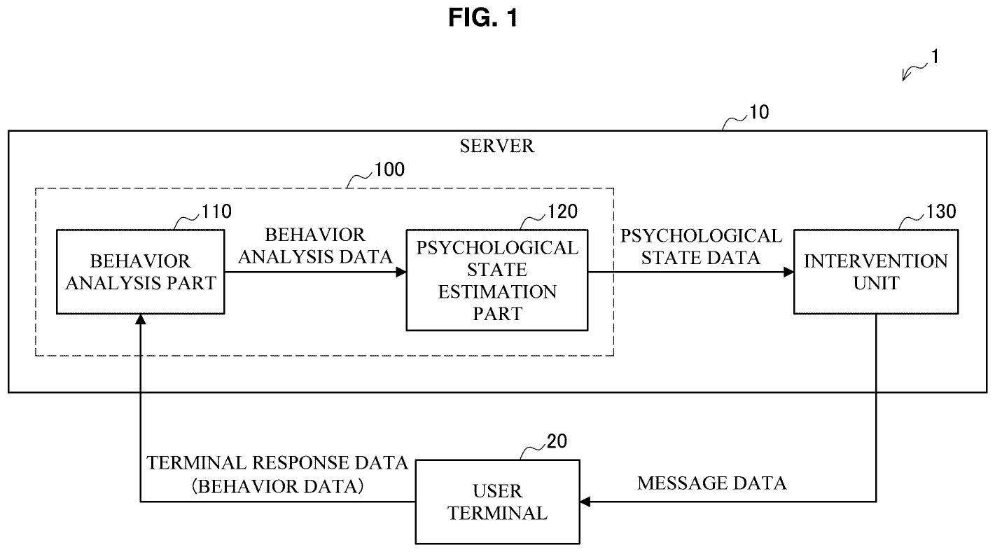

FIG. 1 is a diagram illustrating a configuration example of the psychological state estimation system according to an embodiment of the present invention. As illustrated in FIG. 1, the psychological state estimation system 1 according to the embodiment of the present invention includes a server 10 and a user terminal 20. The server 10 and the user terminal 20 are connected to a network, and configured to be mutually communicable via the network.

### (Server 10)

The server 10 can be achieved by a computer. The server 10 includes a controller (not illustrated) and a storage unit (not illustrated). The controller (not illustrated) includes an estimation unit 100 and an intervention unit 130. The estimation unit 100 includes a behavior analysis part 110 and a psychological state estimation part 120.

Note that the controller (not illustrated) is achieved by a program executed by a processor. Such a program may be recorded on a recording medium, and executed by a processor reading out the program from the recording medium. Alternatively, these blocks may be configured by dedicated hardware.

The storage unit (not illustrated) may be configured by a memory. For example, the memory may be a random access memory (RAM), a hard disk drive, a flash memory, or the like.

### (Estimation Unit 100)

The estimation unit 100 acquires terminal response data output from the user terminal 20. Note that the terminal response data may also correspond to behavior data. The estimation unit 100 estimates psychological state data on the basis of terminal response data. For example, the estimation unit 100 estimates, as psychological state data, at least one of the motivation data, behavior change stage data, and self-efficacy data.

More specifically, the estimation unit 100 estimates motivation data on the basis of service operation data. Moreover, the estimation unit 100 estimates behavior change stage data on the basis of behavior implementation data. The estimation unit 100 estimates self-efficacy data on the basis of behavior implementation data. Note that the service operation data and the behavior implementation data will be described later in detail. Then, the estimation unit 100 outputs psychological state data to the intervention unit 130.

### (Behavior Analysis Part 110)

The behavior analysis part 110 acquires the terminal response data output from the user terminal 20. Then, the behavior analysis part 110 acquires behavior analysis data on the basis of the terminal response data. The behavior analysis part 110 outputs the behavior analysis data to the psychological state estimation part 120.

### (Psychological State Estimation Part 120)

The psychological state estimation part 120 acquires the behavior analysis data output from the behavior analysis part 110. Moreover, the psychological state estimation part 120 estimates psychological state data on the basis of the behavior analysis data output from the behavior analysis part 110. Then, the psychological state estimation part 120 outputs the psychological state data to the intervention unit 130.

### (Intervention Unit 130)

The intervention unit 130 acquires the psychological state data output from the psychological state estimation part 120. Moreover, the intervention unit 130 determines message data on the basis of the psychological state data output from the psychological state estimation part 120. Then, the intervention unit 130 outputs the message data to the user terminal 20.

### (User Terminal 20)

The user terminal 20 may be implemented by a computer. As an example, the user terminal 20 may be a mobile terminal. Having received the message data output from the intervention unit 130, the user terminal 20 outputs terminal response data corresponding to the reception of the message data to the estimation unit 100. For example, the user terminal 20 may be a terminal carried and operated by a user.

### (Service Function)

The user terminal 20 has a service function for the user. For example, the service function may include at least one of an autonomy function, a competence function, a relatedness function, a message viewing function, a message acceptability acquisition function, and a behavior registration function. The service function may be a service function related to stair walking.

### (Autonomy Function)

The autonomy function may be a function of detecting the behavior selected by a user and presenting the details of the behavior selected by the user to the user. For example, the autonomy function may be a function of detecting a user's daily stair use state (for example, the fact that the user went up and down the stairs for three floors yesterday and for two floors the day before yesterday) and presenting the detected stair use state to the user.

### (Competence Function)

The competence function may be a function of presenting excellent results regarding the user's stair use to the user. For example, the excellent result may be information indicating how much the stair use state of the user is superior to that of another user (for example, the information that the amount of stairs going up and down yesterday by the user was one floor more than the amount of stairs going up and down yesterday by another user).

Alternatively, the excellent result may be information indicating how much the user's stair use of today is superior to that of yesterday (for example, the information that the amount of stairs going up and down today by the user was one floor more than the amount of stairs going up and down yesterday by the user). Alternatively, the excellent result may be ranking information in which a plurality of users including the user and other users are ranked in descending order of the amount of stairs going up and down.

### (Relatedness Function)

The relatedness function may be a function of sharing the user's stair use state between the user and another user. For example, the relatedness function may be a function of allowing not only the user but also another user to view the user's stair use state. For example, the stair use state may be shared directly via the network by the user terminal 20 used by the user and the terminal used by another user. The stair use state may be also shared through the server 10 by the user terminal 20 used by the user and the terminal used by another user.

### (Message Viewing Function)

The message viewing function may be a function of presenting the message data output from the intervention unit 130 to the user.

### (Message Acceptability Acquisition Function)

The message acceptability acquisition function may be a function of acquiring the user's acceptability of message data presented to the user (hereinafter, also referred to as “message acceptability”). The message acceptability indicates a user's response in viewing the message data. The following mainly assumes the case in which the message acceptability is selected between two and expressed by either “1: accepted” or “0: not accepted”. However, there may be three or more kinds of candidates for the message acceptability. For example, the message acceptability may be acquired on the basis of user's operation.

### (Behavior Registration Function)

The behavior registration function may be a function of registering the implementation of behavior on the basis of user's operation. For example, the behavior whose implementation is registered by the behavior registration function may be mainly behavior that is difficult to detect using a sensor (for example, eating and the like). For example, the presence or absence of implementation of behavior may be input to a registration page by the user. Alternatively, in a case where the message data includes contents that ask for the selection of presence or absence of implementation of behavior, the presence or absence of implementation of behavior selected by the user may be included in a response to the message data.

The above has described the configuration example of the psychological state estimation system 1 according to the embodiment of the present invention.

### 1-2. Description of Operation

An operation example of the psychological state estimation system 1 according to the embodiment of the present invention will be described.

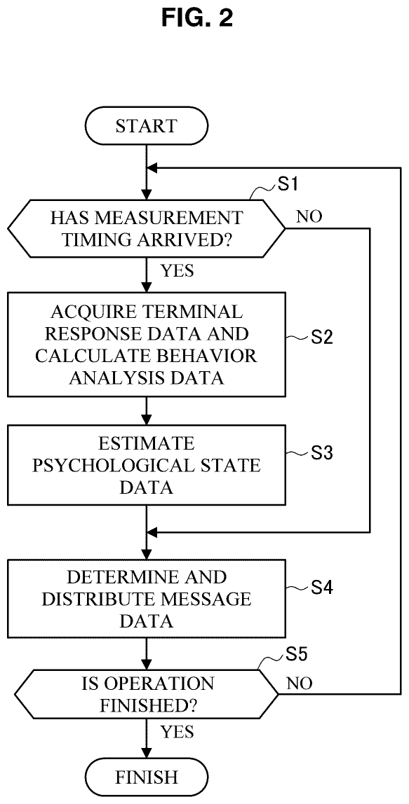

FIG. 2 is a diagram for explaining the operation of the psychological state estimation system 1 according to an embodiment of the present invention. As illustrated in FIG. 2, the operation of the psychological state estimation system 1 according to the embodiment of the present invention is divided to steps of: (S1) Determination of whether the measurement timing has arrived, (S2) Acquisition of terminal response data and calculation of behavior analysis data, (S3) Estimation of psychological state data, (S4) Determination and distribution of message data, and (S5) Determination of whether to finish the operation.

The following will sequentially describe each of these steps.

### (S1) Determination of Whether the Measurement Timing has Arrived

The server 10 determines whether the measurement timing has arrived. For example, the measurement timing arrives at a predetermined cycle. The following mainly assumes the case in which the predetermined cycle is two weeks. However, the predetermined cycle may be arbitrarily set in accordance with the kind of object behavior, the use form of the psychological state estimation system 1, or the like.

In a case where the server 10 determines that the measurement timing has arrived, the processing advances to S2. Note that when the measurement timing has arrived, the current period is between two weeks ago and the present, and the previous period is between four weeks ago and two weeks ago from the present. The current period corresponds to an example of the first period, and the previous period corresponds to an example of the second period, which is the period before the first period. Meanwhile, in a case where the server 10 determines that the measurement timing has not arrived, the processing advances to S4.

### (S2) Acquisition of Terminal Response Data and Calculation of Behavior Analysis Data

The behavior analysis part 110 acquires terminal response data for the current period (between two weeks ago and the present) from the user terminal 20. Then, the behavior analysis part 110 analyzes the acquired terminal response data and acquires behavior analysis data. The behavior analysis part 110 outputs the behavior analysis data to the psychological state estimation part 120, and the processing advances to S3.

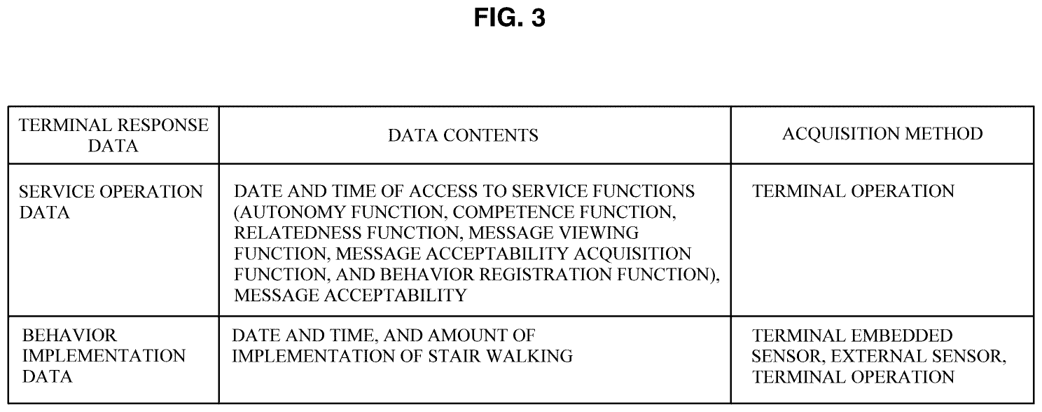

FIG. 3 is a diagram illustrating examples of terminal response data. As illustrated in FIG. 3, the terminal response data includes service operation data and behavior implementation data.

### (Service Operation Data)

The service operation data is user's operation data related to the service function provided to the user by the user terminal 20. As illustrated in FIG. 3, the service operation data includes the date and time of user's access to the service functions (autonomy function, competence function, relatedness function, message viewing function, message acceptability acquisition function, and behavior registration function), the message acceptability, which is user's acceptability of message data, and the like. The service operation data is acquired by operation on the user terminal 20.

### (Behavior Implementation Data)

The behavior implementation data is data indicating when the user implemented stair walking and data indicating how much the user implemented stair walking. That is, as illustrated in FIG. 3, the behavior implementation data may include the date and time of implementation of stair walking and the amount thereof. The behavior implementation data may be acquired by a sensor embedded in the user terminal 20, a sensor provided outside the user terminal 20, on the basis of user's input operation on the user terminal 20, or the like.

For example, the implementation of stair walking may be detected by the user terminal 20 on the basis of the result of atmospheric pressure detection by an atmospheric pressure sensor embedded in the user terminal 20 or the result of acceleration detection by an acceleration sensor embedded in the user terminal 20.

Alternatively, the implementation of stair walking may be detected by the user terminal 20 on the basis of the fact that a receiver embedded in the user terminal 20 has received radio waves from a beacon installed at or near the stairs. Alternatively, the implementation of stair walking may be detected by the user terminal 20 on the basis of the fact that the position detected by a global navigation satellite system (GNSS) sensor embedded in the user terminal 20 belongs to a predetermined stair area.

### (Behavior Analysis Data)

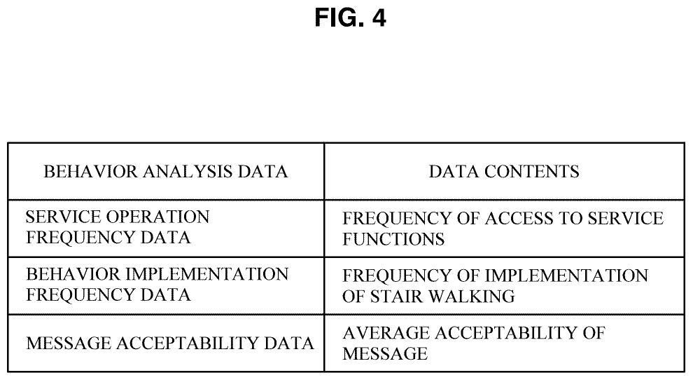

FIG. 4 is a diagram illustrating examples of behavior analysis data. As illustrated in FIG. 4, the behavior analysis data may include service operation frequency data, behavior implementation frequency data, and message acceptability data.

### (Service Operation Frequency Data)

The service operation frequency data is data indicating the frequency of user's access to the service functions, which is calculated by the behavior analysis part 110 on the basis of the service operation data among the terminal response data. For example, the frequency of user's access to the autonomy function is a result of counting the number of times of user's access to the autonomy function during the current period (between two weeks ago and the present) on the basis of the dates and time of user's access to the autonomy function.

### (Behavior Implementation Frequency Data)

The behavior implementation frequency data is data indicating the frequency of implementation of stair walking, which is calculated by the behavior analysis part 110 on the basis of the behavior implementation data among the terminal response data. For example, the frequency of implementation of stair walking is a result of counting the days when the user implemented stair walking of a predetermined amount or more during the current period (between two weeks ago and the present).

### (Message Acceptability Data)

The message acceptability data is data indicating the tendency of message acceptability, which is calculated by the behavior analysis part 110 on the basis of the message acceptability of service operation data among the terminal response data. For example, the tendency of message acceptability may be an average value of the user's message acceptability during the current period (between two weeks ago and the present) (that is, the average acceptability of message). For example, such an average value may be represented by a value equal to or larger than 0 but not exceeding 1.

### (S3) Estimation of Psychological State Data

The psychological state estimation part 120 estimates psychological state data of the present period (between two weeks ago and the present) on the basis of the behavior analysis data output from the behavior analysis part 110. Then, the psychological state estimation part 120 outputs the psychological state data to the intervention unit 130, and the processing advances to S4.

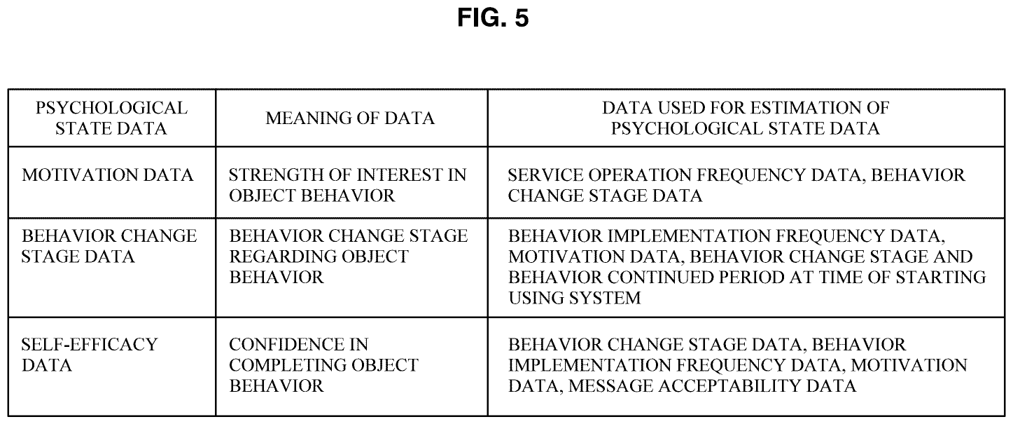

FIG. 5 is a diagram illustrating examples of psychological state data. As shown in FIG. 5, the psychological state data may include motivation data, behavior change stage data, and self-efficacy data. Note that the following mainly assumes that the psychological state estimation part 120 estimates, as psychological state data, all of the motivation data, behavior change stage data, and self-efficacy data. However, the psychological state estimation part 120 may estimate, as psychological state data, at least one of the motivation data, behavior change stage data, and self-efficacy data.

### (Motivation Data)

The motivation data is psychological state data that represents the strength of user's interest in user's stair walking or desire to be achieved by stair walking (for example, diabetes prevention, mood change, and the like). This strength of user's psychological desire for stair walking may vary depending on the service operation frequency data. Then, the psychological state estimation part 120 may estimate the motivation data of the present period (between two weeks ago and the present) on the basis of the service operation frequency data among the behavior analysis data output from the behavior analysis part 110.

For example, the psychological state estimation part 120 may estimate the motivation data to be larger as the service operation frequency data is larger. As an example, the service operation frequency data is divided into 10 levels, and there may be provided correspondence data in which the motivation data of a lower value is sequentially associated in ascending order with the lower level of the service operation frequency data. Here, the psychological state estimation part 120 may estimate the motivation data by acquiring, from the correspondence data, the motivation data associated with the service operation frequency data output from the behavior analysis part 110.

Here, it is mainly assumed that the number of levels of numerical values representing the motivation data is 10. However, the number of levels of numerical values representing the motivation data may not necessarily be 10 levels.

Note that immediately after the user starts using the psychological state estimation system 1, the service operation frequency data may not be calculated. Therefore, the psychological state estimation part 120 may use some data automatically recorded in the user terminal 20 as the motivation data of immediately after the user starts using the psychological state estimation system 1, or as data for calculating the motivation data. Alternatively, the psychological state estimation part 120 may use the motivation data preliminarily acquired from the user as the motivation data of immediately after the user starts using the psychological state estimation system 1.

As an example, there may be preliminarily prepared a table in which the behavior change stage and the motivation data are associated with each other. Here, the user terminal 20 may acquire the current behavior change stage from the user by conducting a questionnaire to the user, and the psychological state estimation part 120 may acquire the motivation data corresponding to the current behavior change stage from the table so as to acquire the motivation data of immediately after the user starts using the psychological state estimation system 1.

### (Behavior Change Stage Data)

The behavior change stage data is psychological state data that represents, by five levels, the states of user's awareness and behavior regarding stair walking. This states of user's awareness and behavior regarding stair walking may vary depending on the behavior implementation frequency data. Then, the psychological state estimation part 120 may estimate the behavior change stage data of the present period (between two weeks ago and the present) on the basis of the behavior implementation frequency data among the behavior analysis data output from the behavior analysis part 110.

More specifically, in the precontemplation period and the contemplation period among the behavior change stages, behavior is hardly implemented. However, in the preparation period, behavior is conducted irregularly. Moreover, in the action period and the maintenance period, behavior is implemented continuously. Then, the psychological state estimation part 120 determines which of these three groups the state of user's awareness and behavior corresponds to, on the basis of the behavior implementation frequency data.

The psychological state estimation part 120 determines whether the number of days of implementation during the current period is smaller than the predetermined first number of days of implementation on the basis of the behavior implementation frequency data. In a case where it is determined that the number of days of implementation during the current period is smaller than the first number of days of implementation, the psychological state estimation part 120 determines that the state of user's awareness and behavior corresponds to the first group: the precontemplation period and the contemplation period.

On the other hand, in a case where it is determined that the number of days of implementation during the current period is equal to or larger than the first number of days of implementation, the psychological state estimation part 120 determines whether the number of days of implementation during the current period is smaller than the predetermined second number of days of implementation. The second number of days of implementation is preliminarily set as the number of days larger than the first number of days of implementation. In a case where it is determined that the number of days of implementation during the current period is smaller than the second number of days of implementation, the psychological state estimation part 120 determines that the state of user's awareness and behavior corresponds to the second group: the preparation period.

In a case where it is determined that the number of days of implementation during the current period is equal to or larger than the second number of days of implementation, the psychological state estimation part 120 determines that the state of user's awareness and behavior corresponds to the third group: the action period and the maintenance period.

Furthermore, a difference between the precontemplation period and the contemplation period is a difference in the degree of interest in stair walking. Therefore, in a case where it is determined that the state of user's awareness and behavior corresponds to the precontemplation period and the contemplation period, the psychological state estimation part 120 determines whether the motivation data of the current period is smaller than a predetermined first threshold.

Then, when it is determined that the motivation data of the current period is smaller than the first threshold, the psychological state estimation part 120 may regard the value indicating the precontemplation period as the behavior change stage data. On the other hand, in a case where it is determined that the motivation data of the current period is equal to or larger than the first threshold, the psychological state estimation part 120 may regard the value indicating the contemplation period as the behavior change stage data.

In addition, a difference between the action period and the maintenance period is a difference in continued period of stair walking. Therefore, in a case where it is determined that the state of user's awareness and behavior corresponds to the action period and the maintenance period, the psychological state estimation part 120 determines whether the continued period of user's stair walking after the action period is smaller than a predetermined second threshold.

Then, in a case where it is determined that the continued period of user's stair walking after the action period is smaller than the second threshold, the psychological state estimation part 120 may regard the value indicating the action period as the behavior change stage data. On the other hand, in a case where it is determined that the continued period of user's stair walking after the action period is equal to or larger than the second threshold, the psychological state estimation part 120 may regard the value indicating the maintenance period as the behavior change stage data.

The continued period of stair walking after the action period may be acquired by any method. As an example, the user terminal 20 may acquire, as initial values, the behavior change stage and the continued period of stair walking at the time when the user starts using the psychological state estimation system 1 on the basis of a questionnaire conducted to the user, and determine, on the basis of these initial values, the continued period of stair walking after the action period, at the time immediately after the user starts using the psychological state estimation system 1.

Thereafter, as long as a period in which the frequency of user's implementation of stair walking is larger than the threshold value continues, on the basis of the behavior implementation frequency data, the psychological state estimation part 120 may add such a period to the initial value of the continued period of stair walking.

Note that immediately after the user starts using the psychological state estimation system 1, the service operation frequency data may not be calculated. Therefore, the psychological state estimation part 120 may use some data automatically recorded in the user terminal 20 as the behavior change stage data of immediately after the user starts using the psychological state estimation system 1, or as data for calculating the behavior change stage data. Alternatively, the psychological state estimation part 120 may use the behavior change stage data preliminarily acquired from the user as the behavior change stage data of immediately after the user starts using the psychological state estimation system 1.

As an example, the user terminal 20 may acquire the current behavior change stage data from the user by conducting a questionnaire to the user, so as to acquire the behavior change stage data of immediately after the user starts using the psychological state estimation system 1.

### (Self-Efficacy Data)

The self-efficacy data is psychological state data that represents the degree of user's confidence in completing stair walking. This degree of user's confidence in completing stair walking may vary depending on the behavior implementation frequency data. Then, the psychological state estimation part 120 may estimate the self-efficacy data of the present period (between two weeks ago and the present) on the basis of the behavior implementation frequency data among the behavior analysis data output from the behavior analysis part 110.

The psychological state estimation part 120 estimates the self-efficacy data on the basis of which of the above-described three groups the behavior change stage indicated by the already-estimated behavior change stage data corresponds to. Note that the following mainly assumes the case in which the self-efficacy data is represented by 10 levels of numerical values. However, the number of levels of numerical values representing the self-efficacy data is not limited to 10 levels.

More specifically, in the precontemplation period and the contemplation period among the behavior change stages, an increase in self-efficacy appears as an increase in motivation, and a decrease in self-efficacy appears as a decrease in motivation. Therefore, the psychological state estimation part 120 may estimate the self-efficacy data of the current period on the basis of the tendency of increase or decrease of motivation data from the previous period (the period between four weeks ago and two weeks ago from the present) to the current period (the period between two weeks ago and the present) and the self-efficacy data of the previous period.

As an example, in a case where the motivation data is increased from the previous period to the current period, the psychological state estimation part 120 may estimate the self-efficacy data of the current period by increasing the self-efficacy data of the previous period by a predetermined increase amount. On the other hand, in a case where the motivation data is decreased from the previous period to the current period, the psychological state estimation part 120 may estimate the self-efficacy data of the current period by decreasing the self-efficacy data of the previous period by a predetermined decrease amount. In a case other than the above-described cases, the psychological state estimation part 120 may use the self-efficacy data of the previous period as the self-efficacy data of the current period.

Note that in the precontemplation period and the contemplation period among the behavior change stages, an increase in self-efficacy may also appear as an increase in frequency of behavior. Therefore, the psychological state estimation part 120 may add a condition that the behavior implementation frequency data tends to be increased from the previous period to the current period as the condition for increasing the self-efficacy data. That is, only in a case where both the motivation data and the behavior implementation frequency data are increased from the previous period to the current period, the psychological state estimation part 120 may increase the self-efficacy data of the previous period by a predetermined increase amount.

In the preparation period among the behavior change stages, an increase in self-efficacy appears as an increase in frequency of behavior or an increase in motivation, and a decrease in self-efficacy appears as a decrease in frequency of behavior or a decrease in motivation. Therefore, the psychological state estimation part 120 may estimate the self-efficacy data of the current period on the basis of the tendency of increase or decrease of at least one of the motivation data and the behavior implementation frequency data from the previous period to the current period and the self-efficacy data of the previous period.

As an example, in a case where one of the motivation data and the behavior implementation frequency data or both of them are increased from the previous period to the current period, the psychological state estimation part 120 may estimate the self-efficacy data of the current period by increasing the self-efficacy data of the previous period by a predetermined increase amount. On the other hand, in a case where none of the motivation data and the behavior implementation frequency data is increased and one of the motivation data and the behavior implementation frequency data or both of them are decreased from the previous period to the current period, the psychological state estimation part 120 may estimate the self-efficacy data of the current period by decreasing the self-efficacy data of the previous period by a predetermined decrease amount. In a case other than the above-described cases, the psychological state estimation part 120 may use the self-efficacy data of the previous period as the self-efficacy data of the current period.

In the action period and the maintenance period among the behavior change stages, an increase in self-efficacy appears as the maintenance or an increase in frequency of behavior, and a decrease in self-efficacy appears as a decrease in frequency of behavior. Therefore, the psychological state estimation part 120 may estimate the self-efficacy data of the current period on the basis of the tendency of increase or decrease of the behavior implementation frequency data from the previous period to the current period and the self-efficacy data of the previous period.

As an example, in a case where the behavior implementation frequency data is increased from the previous period to the current period, the psychological state estimation part 120 may estimate the self-efficacy data of the current period by increasing the self-efficacy data of the previous period by a predetermined increase amount. On the other hand, in a case where the behavior implementation frequency data is decreased from the previous period to the current period, the psychological state estimation part 120 may estimate the self-efficacy data of the current period by decreasing the self-efficacy data of the previous period by a predetermined decrease amount. In a case other than the above-described cases, the psychological state estimation part 120 may use the self-efficacy data of the previous period as the self-efficacy data of the current period.

Moreover, in a case where the user's behavior implementation frequency data of the current period is smaller than a predetermined first value, and/or in a case where the user's message acceptability data of the current period is smaller than a predetermined second value, the psychological state estimation part 120 may make correction for decreasing the self-efficacy data. The first value may be an arbitrarily set value, and may be 8 days/2 weeks, for example. The second value may be also an arbitrarily set value, and may be 0.7, for example.

In this manner, even if a message difficult for the user to accept (for example, recommendation for difficult behavior, and the like) is presented to the user, the psychological state estimation part 120 makes correction for decreasing the self-efficacy data, so as to allow the intervention unit 130 to determine a message easier for the user to accept on the basis of such self-efficacy data. The operation of the intervention unit 130 will be described at Step S4.

Note that immediately after the user starts using the psychological state estimation system 1, the service operation frequency data may not be calculated. Therefore, the psychological state estimation part 120 may use some data automatically recorded in the user terminal 20 as the self-efficacy data of immediately after the user starts using the psychological state estimation system 1, or as data for calculating the self-efficacy data. Alternatively, the psychological state estimation part 120 may use the self-efficacy data preliminarily acquired from the user as the self-efficacy data of immediately after the user starts using the psychological state estimation system 1.

As an example, a table in which the behavior change stage and the self-efficacy data are associated with each other may be preliminarily prepared. Here, the user terminal 20 may acquire the current behavior change stage from the user by conducting a questionnaire to the user, and the psychological state estimation part 120 may acquire the self-efficacy data corresponding to the current behavior change stage from the table so as to acquire the self-efficacy data of immediately after the user starts using the psychological state estimation system 1.

### (S4) Determination and Distribution of Message Data

The intervention unit 130 intervenes in user's stair walking. Specifically, the intervention unit 130 determines message data on the basis of the psychological state data output from the psychological state estimation part 120. Then, the intervention unit 130 intervenes by outputting the determined message data to the user terminal 20, and the processing advances to Step S5. The following will specifically describe a method for determining message data.

First, the intervention unit 130 preliminarily holds a group of message data to be provided to the user for each psychological state data (motivation data and behavior change stage data) and existing habit. The existing habit may include information indicating what kind of behavior the user implements under what kind of environmental condition. For example, the environmental condition may include conditions related to a time slot, location and weather. Here, the intervention unit 130 holds a group of message data in a state where the behavior level corresponding to a difficulty level of user's message acceptance is linked to each message data. Note that the behavior level may indicate the strength or difficulty level of behavior.

For example, the behavior level is represented by numerical values of seven levels from 1 to 7. However, the number of levels of numerical values representing the behavior level is not limited to seven.

Moreover, the intervention unit 130 holds the self-efficacy data at the time when the user starts using the psychological state estimation system 1 (hereinafter, also referred to as “past self-efficacy data”) and the behavior level at such time (hereinafter, also referred to as “past behavior level”), and the user's target behavior level.

The intervention unit 130 may acquire past self-efficacy data by any method. For example, the intervention unit 130 may acquire past self-efficacy data input to the user terminal 20 by the user immediately after the user starts using the psychological state estimation system 1. Moreover, the intervention unit 130 may acquire a past behavior level and a target behavior level by any method. For example, the intervention unit 130 may acquire a past behavior level and a target behavior level that are input to the user terminal 20 by the user when the user starts using the psychological state estimation system 1.

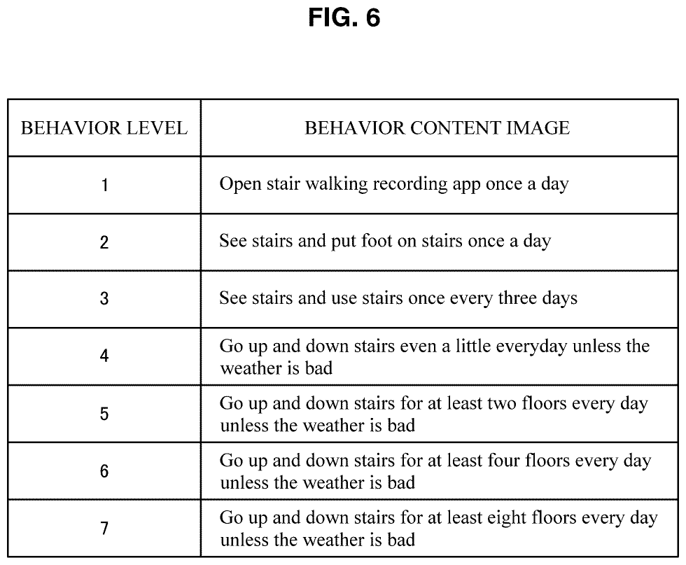

FIG. 6 is a diagram illustrating examples of behavior levels and behavior content images. When the user inputs a past behavior level and a target behavior level to the user terminal 20, it is preferable that the user terminal 20 presents to the user a behavior content image for each behavior level as exemplified in FIG. 6. This allows the user to easily select a current behavior level and a target behavior level.

The intervention unit 130 estimates a behavior level suitable for the user on the basis of the fact that the psychological state data has been output from the psychological state estimation part 120. More specifically, the intervention unit 130 estimates a behavior level suitable for the user on the basis of the past behavior level and the target behavior level that are input by the user, the past self-efficacy data, and the self-efficacy data estimated by the psychological state estimation part 120. As a specific method for estimating a behavior level, it is possible to adopt various estimation methods.

For example, the intervention unit 130 may use four kinds of data: self-efficacy data (represented as SE) estimated by the psychological state estimation part 120, preliminarily held past self-efficacy data (represented as SE0), a past behavior level (represented as L0), and a target behavior level (represented as LG), so as to estimate a behavior level suitable for the user (represented by L) according to the linear prediction formula shown in the following formula (1):

$$L = ( L G - L 0 ) / ( S E max - SE 0 ) \times ( S E - SE 0 ) + L 0 \qquad ( 1 )$$

Here, SEmax represents the maximum value of self-efficacy data. In a case where the self-efficacy data is represented by numerical values of 10 levels from 1 to 10, SEmax=10 is established.

In the above-described formula (1), the accomplishable behavior level at the time when the user starts using the psychological state estimation system 1 is set as an initial value. The behavior level is gradually increased as the self-efficacy data is increased, and is calculated to reach the user's target behavior level when the self-efficacy data reaches the maximum. Therefore, it is possible to estimate a behavior level that is easy to accomplish in accordance with the degree of user's self-efficacy.

The intervention unit 130 determines message data corresponding to the estimated behavior level, from the preliminarily held group of message data. For example, the intervention unit 130 determines message data corresponding to the estimated behavior level, the currently met environmental conditions, and the current behavior of the user, from the preliminarily held group of message data.

The intervention unit 130 outputs the determined message data to the user terminal 20, so that the user terminal 20 displays the message data. The user notices the message data displayed by the user terminal 20, and changes the awareness and behavior regarding stair walking.

Although omitted from the configuration diagram illustrated in FIG. 1, the intervention unit 130 may detect a time slot, location, weather, and user's behavior on the basis of the behavior implementation data or the like among the terminal response data output by the user terminal 20 in order to determine what kind of environmental condition is currently met and what kind of behavior the user currently implements.

Specifically, on the basis of the date and time of implementation of stair walking included in the behavior implementation data, the intervention unit 130 may detect a time slot to which such date and time belong. Moreover, on the basis of the position detected by the GNSS sensor embedded in the user terminal 20, the intervention unit 130 may determine a location to which such a position belongs. The intervention unit 130 may also acquire weather corresponding to such a position from an external weather service. Furthermore, the intervention unit 130 may detect user's behavior on the basis of a result of acceleration detection by an acceleration sensor embedded in the user terminal 20.

In this manner, the intervention unit 130 may detect a situation that “a user of a specific behavior level passes an elevator hall when moving in a building during the daytime on weekdays”, and output message data corresponding to this situation “It is good for your health to go up and down the stairs even a little every day” to the user terminal 20.

Moreover, the intervention unit 130 may determine message data on the basis of the motivation data and/or behavior change stage data among the psychological state data output from the psychological state estimation part 120.

For example, in a case where the user's motivation data is large, it may be assumed that the user's acceptability to message data is high. Therefore, as the user's motivation data is larger, the intervention unit 130 may output message data to the user terminal 20 at a higher frequency by relaxing the conditions for outputting message data. Moreover, the intervention unit 130 may change the content of message data in accordance with the behavior change stage data and output the message data to the user terminal 20.

Note that in a case where no message data is extracted from the group of message data, the intervention unit 130 does not output anything to the user terminal 20, and the processing advances to S5.

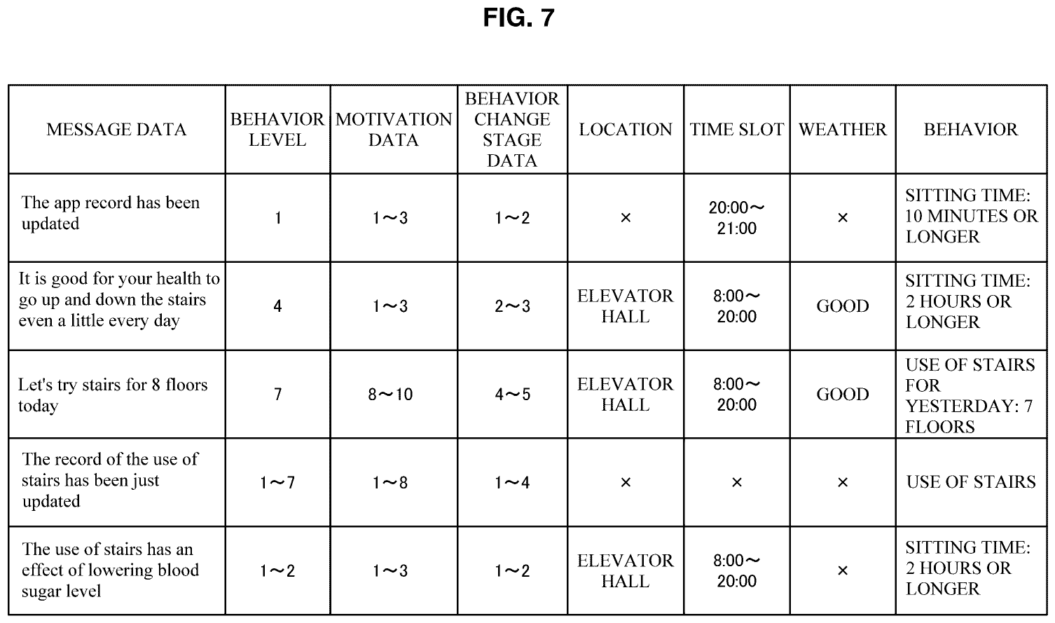

FIG. 7 illustrates examples of message data held by the intervention unit 130. As described above, the behavior level, the psychological state data (motivation data and behavior change stage data), the environmental conditions (conditions related to a time slot, location, weather), and user's behavior are defined for each message data, as conditions for outputting the message data.

For example, FIG. 7 illustrates the example in which the conditions for outputting the message data “It is good for your health to go up and down the stairs even a little every day” are: the behavior level is 4, the motivation data is 1 to 3, the behavior change stage data is 2 to 3 (contemplation period or preparation period), the user's location is an elevator hall, the time slot is 8:00 to 20:00, the weather is good, and the user's sitting time is two hours or longer.

### (S5) Determination of Whether to Finish the Operation

When the user inputs operation for finishing the use to the user terminal 20, the operation of the psychological state estimation system 1 is finished. Otherwise, the processing is shifted to S1.

The operation example of the psychological state estimation system 1 according to the embodiment of the present invention has been described.

### 1-3. Explanation of Effect

As described above, in the psychological state estimation system 1 according to the embodiment of the present invention, the behavior analysis part 110 inputs terminal response data indicating a user's behavior result from the user terminal 20, and outputs behavior analysis data. Then, on the basis of the behavior analysis data, the psychological state estimation part 120 outputs, as psychological state data, at least one of the motivation data, behavior change stage data, and self-efficacy data.

The intervention unit 130 outputs message data suitable for the psychological state data to the user terminal 20. Here, the user may view the message data displayed by the user terminal 20 and implement behavior.

The psychological state data is estimated by the psychological state estimation part 120 on the basis of the terminal response data output from the user terminal 20 and, more specifically, on the basis of the sensor data or service operation data automatically collected by the user terminal 20. This exerts the effects of reducing the workload for the user and easily grasping how the user's psychological state changes with time.

Furthermore, the psychological state estimation system 1 according to the embodiment of the present invention may estimate, as psychological state data, not only the behavior change stage data but also self-efficacy data and motivation data. This exerts the effect of accurately grasping the psychological state of the user in the first half stage of behavior change. The psychological state in such a stage is difficult to grasp only with the behavior change stage data.

As described above, the embodiment of the present invention is capable of solving the problem that the temporal change in user's psychological state cannot be grasped easily and accurately, and acquiring the user's psychological progress of behavior change easily and accurately, including the user who is in the first half stage of behavior change.

The details of the embodiment of the present invention have been described.

### 2. Hardware Configuration Example

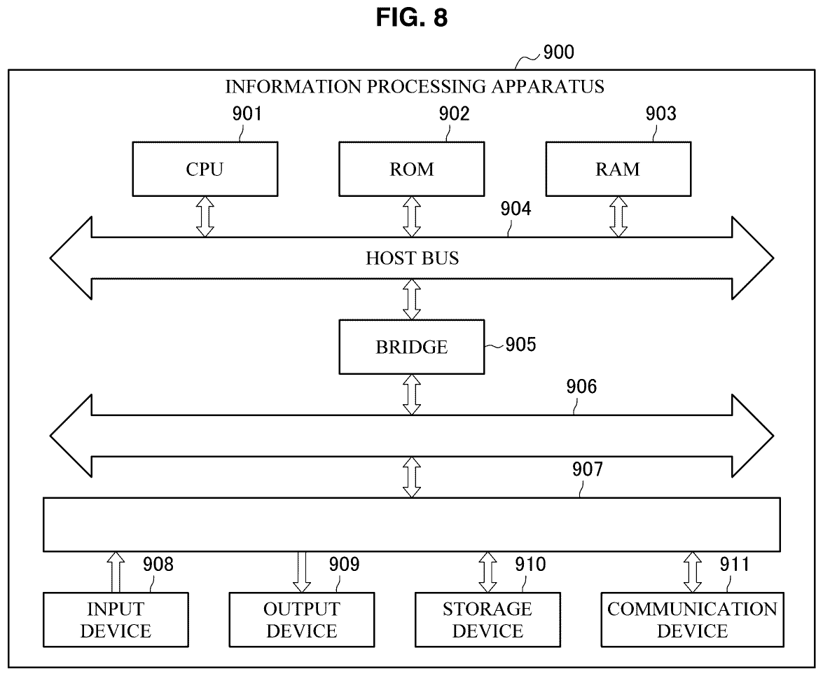

The following will describe a hardware configuration of an information processing apparatus 900 as an example of the hardware configuration of the server 10 according to an embodiment of the present invention. FIG. 8 is a diagram illustrating a hardware configuration of the information processing apparatus 900 as an example of the hardware configuration of the server 10 according to an embodiment of the present invention. Note that the hardware configuration of the user terminal 20 may be also realized in the same manner as the hardware configuration of the information processing apparatus 900 as illustrated in FIG. 8.

As illustrated in FIG. 8, the information processing apparatus 900 includes a central processing unit (CPU) 901, a read only memory (ROM) 902, a random access memory (RAM) 903, a host bus 904, a bridge 905, an external bus 906, an interface 907, an input device 908, an output device 909, a storage device 910, and a communication device 911.

The CPU 901 functions as an arithmetic processing device and a control device, and controls the overall operation in the information processing apparatus 900 in accordance with various programs. Moreover, the CPU 901 may be a microprocessor. The ROM 902 stores programs, parameters, and the like used by the CPU 901. The RAM 903 temporarily stores programs used in the execution of the CPU 901, parameters changing appropriately in the execution thereof, and the like. These are mutually connected by the bus 904 formed by a CPU bus or the like.

The host bus 904 is connected to the external bus 906 such as a peripheral component interconnect/interface (PCI) bus via the bridge 905. Note that it is not necessarily required to configure the host bus 904, the bridge 905, and the external bus 906 separately and these functions may be implemented on one bus.

The input device 908 includes input means for the user to input information, such as a mouse, keyboard, touch panel, button, microphone, switch, and lever, an input control circuit that generates an input signal on the basis of the user's input and outputs it to the CPU 901, and the like. With the operation on the input device 908, the user who operates the information processing apparatus 900 is able to input various kinds of data to the information processing apparatus 900, or instruct the information processing apparatus 900 to perform processing operation.

Examples of the output device 909 include, for example, a cathode ray tube (CRT) display device, a liquid crystal display (LCD) device, an organic light emitting diode (OLED) device, a display device such as a lamp, and an audio output device such as a speaker.

The storage device 910 is a device for storing data. The storage device 910 may include a storage medium, a recording device that records data on a storage medium, a reading device that reads data from a storage medium, a deletion device that deletes data recorded on a storage medium, and the like. The storage device 910 includes, for example, a hard disk drive (HDD). The storage device 910 drives a hard disk, and stores programs and various kinds of data executed by the CPU 901.

The communication device 911 is, for example, a communication interface formed by a communication device for connection to a network, or the like. Moreover, the communication device 911 may support either wireless communication or wired communication.

The above has described the example of the hardware configuration of the information processing apparatus 900 as an example of the server 10 according to the embodiment of the present invention.

### 3. Modifications

Heretofore, preferred embodiments of the present invention have been described in detail with reference to the appended drawings, but the present invention is not limited thereto. It is obvious that a person skilled in the art can arrive at various alterations and changes within the scope of the technical ideas defined in the claims, and it should be naturally understood that such alterations and changes are also encompassed by the technical scope of the present invention.

### 3-1. Modification Related to Object Behavior

The above has described the case in which the user makes stair walking, as an example of the object behavior, a habit. However, the object behavior is not limited to stair walking.

For example, the object behavior may be walking, eating, exercising, interacting, learning, environmentally friendly behavior, or the like. Here, if the kind of terminal response data is defined in accordance with the type of object behavior, it is possible to exert the same effects as those in the case where the object behavior is stair walking. For example, in a case where the object behavior is environmentally friendly behavior, the amount of greenhouse gas in a purchased product may be defined as the terminal response data.

### 3-2. Modification Related to System Configuration

The above has mainly described the case in which the behavior analysis part 110, the psychological state estimation part 120, and the intervention unit 130 are provided in the server 10, and the user terminal 20 is a mobile terminal. However, the configuration of the psychological state estimation system 1 is not limited to such an example.

For example, all of the behavior analysis part 110, the psychological state estimation part 120, and the intervention unit 130, or a part of them may be provided in the user terminal 20, and the user terminal 20 may be a signage terminal, a smart speaker, a communication robot, or the like. Moreover, it is not necessary that all of the terminal response data input to the behavior analysis part 110 be acquired from the user terminal 20. For example, a camera system installed in a building may capture an image of a user, and output behavior data of the user recognized from the image captured by the camera system to the behavior analysis part 110.

### 3-3. Modification Related to Psychological State Data

The above has mainly described the case in which the psychological state estimation part 120 estimates, as psychological state data, the motivation data, behavior change stage data, and self-efficacy data. As described in the following, such motivation data, behavior change stage data, and self-efficacy data may be interpreted in a broad sense.

For example, the “motivation” indicates the strength of user's interest in object behavior or desire to be achieved by object behavior. Therefore, the “motivation” may broadly include senses of “interest in object behavior”, “intensity of desire”, “intensity of pain”, and the like.

Moreover, the “behavior change stage” generally represents, by five levels, the state of subject's awareness and behavior regarding the object behavior. Therefore, the “behavior change stage” may broadly include the states of awareness and behavior estimated using an index classified by the number of levels other than five levels.

Furthermore, the “self-efficacy” indicates the degree of user's confidence in completing object behavior. Therefore, the “self-efficacy” may broadly include senses of “confidence in behavior”, “easiness of execution of behavior”, “ability to implement behavior”, and the like.

### 3-4. Modification Related to Estimation of Behavior Level

The above has mainly described the example in which the intervention unit 130 estimates a behavior level by the linear prediction formula based on user's self-efficacy data and the message data suitable for the estimated behavior level is output. However, the method for estimating a behavior level may not be limited to the above-described example using the linear prediction formula.

For example, in a case where the relation between the self-efficacy data and the behavior level is experimentally found in advance and the relation therebetween is expressed in a table form, a behavior level may be estimated from such relation therebetween and the self-efficacy data.

What is claimed is:

1. A psychological state estimation system, comprising:
an estimation unit configured to estimate, as psychological state data of a subject, at least one of motivation data, behavior change stage data, and self-efficacy data that are related to object behavior of the subject, on the basis of behavior data of the subject along time series.

2. The psychological state estimation system according to claim 1, further comprising:
an intervention unit configured to intervene in the object behavior of the subject, wherein
the intervention unit intervenes by determining message data on the basis of the psychological state data and outputting the message data to a terminal of the subject.

3. The psychological state estimation system according to claim 2, wherein the intervention unit estimates a behavior level indicating a strength of the object behavior suitable for the subject, on the basis of the self-efficacy data, and determines message data corresponding to the behavior level.

4. The psychological state estimation system according to claim 3, wherein the intervention unit estimates the behavior level on the basis of a past behavior level and a target behavior level that are input by the subject, past self-efficacy data, and the self-efficacy data estimated by the estimation unit.

5. The psychological state estimation system according to claim 3, wherein
the estimation unit makes correction for decreasing the self-efficacy data in a case where subjects' behavior implementation frequency data of a first period is smaller than a predetermined first value, the behavior implementation frequency data indicating the number of days of implementation of the object behavior, and/or in a case where subject's message acceptability data regarding message data output to the terminal of the subject in the first period is smaller than a predetermined second value, and
the intervention unit estimates the behavior level on the basis of self-efficacy data after the correction.

6. The psychological state estimation system according to claim 1, wherein
the behavior data includes service operation data that is operation data of the subject related to a service function for the subject, and
the estimation unit estimates the motivation data on the basis of the service operation data.

7. The psychological state estimation system according to claim 6, wherein
the estimation unit includes:
a behavior analysis part configured to calculate service operation frequency data indicating a frequency of access to the service function by the subject during a first period, on the basis of the service operation data; and
a psychological state estimation part configured to estimate motivation data of the first period on the basis of the service operation frequency data.

8. The psychological state estimation system according to claim 7, wherein the psychological state estimation part estimates the motivation data of the first period to be larger as the service operation frequency data is larger.

9. The psychological state estimation system according to claim 1, wherein
the behavior data includes behavior implementation data including data indicating when the subject implemented the object behavior, and
the estimation unit estimates the behavior change stage data on the basis of the behavior implementation data.

10. The psychological state estimation system according to claim 9, wherein
the estimation unit includes:
a behavior analysis part configured to calculate behavior implementation frequency data indicating the number of days of implementation of the object behavior by the subject during a first period, on the basis of the behavior implementation data; and
a psychological state estimation part configured to estimate behavior change stage data of the first period on the basis of the behavior implementation frequency data.

11. The psychological state estimation system according to claim 10, wherein
the estimation unit estimates the motivation data, and
the psychological state estimation part determines whether the number of days of implementation during the first period is smaller than a first number of days of implementation on the basis of the behavior implementation frequency data, determines, in a case where it is determined that the number of days of implementation during the first period is smaller than the first number of days of implementation, whether the motivation data is smaller than a first threshold, regards a value indicating a precontemplation period as the behavior change stage data in a case where it is determined that the motivation data is smaller than the first threshold, and regards a value indicating a contemplation period as the behavior change stage data in a case where it is determined that the motivation data is equal to or larger than the first threshold.

12. The psychological state estimation system according to claim 10, wherein the psychological state estimation part determines whether the number of days of implementation during the first period is equal to or larger than the first number of days of implementation and smaller than a second number of days of implementation on the basis of the behavior implementation frequency data, and regards a value indicating a preparation period as the behavior change stage data in a case where it is determined that the number of days of implementation during the first period is equal to or larger than the first number of days of implementation and smaller than the second number of days of implementation.

13. The psychological state estimation system according to claim 10, wherein the psychological state estimation part determines whether the number of days of implementation during the first period is equal to or larger than the second number of days of implementation on the basis of the behavior implementation frequency data, determines, in a case where it is determined that the number of days of implementation during the first period is equal to or larger than the second number of days of implementation, whether a continued period after an action period of the object behavior by the subject is smaller than a second threshold, regards a value indicating an action period as the behavior change stage data in a case where it is determined that the continued period after the action period is smaller than the second threshold, and regards a value indicating a maintenance period as the behavior change stage data in a case where it is determined that the continued period after the action period is equal to or larger than the second threshold.

14. The psychological state estimation system according to claim 1, wherein
the behavior data includes behavior implementation data including data indicating when the subject implemented the object behavior, and
the estimation unit estimates the self-efficacy data on the basis of the behavior implementation data.

15. The psychological state estimation system according to claim 14, wherein
the estimation unit includes:
a behavior analysis part configured to calculate behavior implementation frequency data indicating the number of days of implementation of the object behavior by the subject during a first period, on the basis of the behavior implementation data; and
a psychological state estimation part configured to estimate the self-efficacy data of the first period on the basis of the behavior implementation frequency data.

16. The psychological state estimation system according to claim 15, wherein
the estimation unit estimates the motivation data, and
the psychological state estimation part determines whether the number of days of implementation during the first period is smaller than a first number of days of implementation on the basis of the behavior implementation frequency data, and estimates, in a case where it is determined that the number of days of implementation during the first period is smaller than the first number of days of implementation, self-efficacy data of the first period on the basis of a tendency of increase or decrease of motivation data from a second period that is a period before the first period to the first period and the self-efficacy data of the second period.

17. The psychological state estimation system according to claim 15, wherein
the estimation unit estimates the motivation data, and
the psychological state estimation part determines whether the number of days of implementation during the first period is equal to or larger than a first number of days of implementation and smaller than a second number of days of implementation on the basis of the behavior implementation frequency data, and estimates, in a case where it is determined that the number of days of implementation during the first period is equal to or larger than the first number of days of implementation and smaller than the second number of days of implementation, self-efficacy data of the first period on the basis of a tendency of increase or decrease of at least one of motivation data and behavior implementation frequency data from a second period that is a period before the first period to the first period and the self-efficacy data of the second period.

18. The psychological state estimation system according to claim 15, wherein
the psychological state estimation part determines whether the number of days of implementation during the first period is equal to or larger than the second number of days of implementation on the basis of the behavior implementation frequency data, and estimates, in a case where it is determined that the number of days of implementation during the first period is equal to or larger than the second number of days of implementation, self-efficacy data of the first period on the basis of a tendency of increase or decrease of the behavior implementation frequency data from a second period that is a period before the first period to the first period and the self-efficacy data of the second period.

19. A psychological state estimation method, comprising:
Estimating by a processor, as psychological state data of a subject, at least one of motivation data, behavior change stage data, and self-efficacy data that are related to object behavior of the subject, on the basis of behavior data of the subject along time series.

20. A non-transitory computer readable storage medium recording a program, the program causing a computer to function as:
an estimation unit configured to estimate, as psychological state data of a subject, at least one of motivation data, behavior change stage data, and self-efficacy data that are related to object behavior of the subject, on the basis of behavior data of the subject along time series.
